(19) United States
(12) Patent Application Publication
(10) Pub. No.: US 2004/0091914 A1
(43) Pub. Date: May 13, 2004

(54) Gene assay method for predicting glaucoma onset risk

(75) Inventors: Yasuhiro Kouchi, Kobe-shi (JP); Akinori Masago, Kobe-shi (JP); Takayuki Takahata, Kobe-shi (JP)

Correspondence Address: SUGHRUE MION, PLLC, 2100 PENNSYLVANIA AVENUE, N.W., SUITE 800, WASHINGTON, DC, 20037, US

(73) Assignee: SYSMEX CORPORATION

(21) Appl. No.: 10/627,757

(22) Filed: Jul. 28, 2003

(30) Foreign Application Priority Data
Aug. 2, 2002 (JP) JP 2002-226612

(51) Int. Cl. C12Q 1/68; C07H 21/04

(52) U.S. Cl. 435/6; 536/24.3

(57) ABSTRACT

Future onset of glaucoma is predicted using as a marker, mutation of base(s) in a coding region of a glaucoma-related gene(OPTN gene).

### BACKGROUND OF THE INVENTION

1. Field of the Invention

The present invention relates to an assay method for a glaucoma-related gene in the clinical testing field, and to an assay method for predicting a glaucoma onset risk using a mutation(s) in the gene as a marker. More specifically, the invention relates to a gene assay method in which, for example, a mutation of the gene encoding optineurin (hereinafter referred to as “OPTN”) known as a glaucoma-related gene, is detected. Glaucoma is thereby predicted using, as a marker, the detected anomaly, i.e., a mutation of a base or bases at a specific position or specific positions in the gene. More particularly, the present invention relates to an assay method for predicting the possibility of a future onset of glaucoma in an individual.

2. Description of the Related Art

Glaucoma is a disease in which it is difficult to discharge aqueous humor from the eye and as a result ocular tension increases, resulting in a degradation of the functions of the eye. If a glaucomatous eye is left untreated, the field of vision of the eye becomes smaller or eyesight is degraded, eventually resulting in loss of eyesight. There are also cases where the ocular tension is normal but the optic nerve is damaged.

Glaucoma is classified in five disease types such as primary open angle glaucoma (POAG), normal ocular tension glaucoma (NTG), primary closing angle glaucoma (PCAG), congenital glaucoma and secondary glaucoma. Among the 20% of glaucoma said to be genetic, POAG is found in most cases. According to a nationwide epidemiological survey conducted by Tapan Oculists' Society, an incorporated body, 3.56% of the population of those who are forty years old or older are glaucoma patients.

The major risk factor of glaucoma is familial anamnesis and it is strongly suggested that the onset of glaucoma has a genetic basis. In U.S. Pat. No. 5,789,169 (Nguyen et al.) filed on May 17, 1996, a gene that encodes TIGR (trabecular meshwork-induced glucocorticoid response) protein is disclosed as a glaucoma related gene. The TIGR gene is also known as the MYOC gene. The U.S. Pat. No. 5,789,169 (Nguyen et al.) also discloses the cDNA sequence that encodes the TIGR protein, the protein itself, molecules bonded to the protein and the nucleic acid molecule encoding the bonded molecules. That patent further provides a method and a reagent for diagnosing glaucoma and glaucoma-related diseases, as well as other diseases such as diseases of cardiac blood vessels, immune diseases, and diseases influenced by expression and activity of said protein. A method of diagnosing glaucoma in an individual by detecting the presence of a mutation in the CYP1B1 gene, one of the glaucoma-related genes that may be used as a marker of glaucoma is also disclosed (International Patent Publication No. WO 98/36098 A1). Furthermore, Rezaie, T. et al. identified the OPTN (optinuerin) gene as causing glaucoma within the GLC1E region (Science, Feb. 8, 2002; 295 (5557): 1077-9). The OPTN gene (Genbank entry NM_{—}021980) was previously known as the FIP2 gene. However, means to predict the onset risk of glaucoma in the future are not disclosed in these publications.

On the other hand, in International Patent Publication No. WO 01/88120 A1, a method of detecting a mutation at position −153 of the promoter region of the MYOC gene is disclosed. It is also described that the method can be used as a screening method for glaucoma for a patient worrying that he/she is in a family line that has a heredity risk of glaucoma, or he/she is a glaucoma carrier having no developed symptoms.

However, OPTN gene is not disclosed in the WO 01/88120A1. Since glaucoma develops slowly, a better method of early diagnosis or effectively predicting the possibility of an onset of glaucoma is desired so that measures for preventing or alleviating glaucoma can be taken before serious damage is caused to the optic nerve.

If individuals possessing a genetic risk of developing glaucoma can be identified, and examination for glaucoma can be concentrated on such individuals, it is considered that early detection and early treatment of glaucoma can be conducted efficiently. Concerning such a situation, it is the purpose of this invention to provide an assay method for genes that can be used to effectively predict the onset risk of glaucoma based upon studying the relation between glaucoma-related genes and the onset of glaucoma.

### SUMMARY OF THE INVENTION

Noting that the onset of glaucoma is related to a mutation of a gene(s), the inventors analyzed the gene sequence of the coding region of OPTN genes of glaucoma patients and non-patients and made an effort at studying them. As a result, the inventors found that the frequencies of a mutation observed for the gene(s) are significantly different between the group of patients and the group of non-patients. Furthermore, the inventors found that the onset rate of glaucoma has statistically significant dependence on whether the mutation is present or not, compared to the onset rate of the general group, and the inventors thus completed the invention.

That is, the present invention provides:

1. A gene assay method comprising the steps of: detecting a mutation of at least one base in the coding region of an optineurin (OPTN) gene of a human subject; and predicting future onset of glaucoma in the subject using the mutation as an index.

2. The gene assay method of item1, wherein the coding region of said glaucoma-related gene is an OPTN gene has a nucleic acid sequence denoted by SEQ ID NO: 1.

3. The gene assay method of item 2, wherein said mutation is a substitution of G for A at position 619 and/or a substitution of A for G at position 898 in the nucleic acid sequence denoted by SEQ ID NO: 1.

4. The gene assay method of item 2, wherein said mutation is a deletion of one or more bases in the nucleic acid sequence denoted by SEQ ID NO: 1.

5. The gene assay method of item 2, wherein said mutation is an insertion of one or more bases in the nucleic acid sequence denoted by SEQ ID NO: 1.

6. The gene assay method of item 2, wherein said mutation is two or more substitutions of bases in the nucleic acid sequence denoted by SEQ ID NO: 1.

7. The gene assay method according to claim 1, wherein the glaucoma is primary open angle glaucoma and/or normal ocular tension glaucoma.

8. The gene assay method according to item 1, wherein the mutation is detected by using an oligonucleotide capable of forming a hybrid at a specific position of the coding region of the OPTN gene.

9. An oligonucleotide selected from the group consisting of oligonucleotides comprising sequences as follows:

(1) an oligonucleotide consisting of a base sequence represented by any of SEQ ID NOs: 15 to 40;

(2) a complementary chain of an oligonucleotide according to (1);

(3) an oligonucleotide that hybridizes with an oligonucleotide according to (1) or (2) under stringent conditions;

(4) an oligonucleotide having a homology of 60% or more to an oligonucleotide according to any one of (1) to (3); and

(5) an oligonucleotide according to any one of (1) to (4) having one to several bases mutated by substitution, deletion, insertion or addition.

10. A gene assay method for predicting future onset of primary open angle glaucoma and/or normal ocular tension glaucoma, comprising the steps of:

(a) isolating a polynucleotide sample from a subject suspected of having a mutation in a glaucoma-related gene,

(b) performing a nucleic acid amplification process on said polynucleotide using at least one oligonucleotide selected from the group consisting of oligonucleotides comprising sequences as follows:

(1) an oligonucleotide consisting of a base sequence represented by any of SEQ ID NOs: 15 to 40;

(2) a complementary chain of an oligonucleotide according to (1);

(3) an oligonucleotide that hybridizes with an oligonucleotide according to (1) or (2) under stringent conditions;

(4) an oligonucleotide having a homology of 60% or more to an oligonucleotide according to any one of (1) to (3); and

(5) an oligonucleotide according to any one of (1) to (4) having one to several bases mutated by substitution, deletion, insertion or addition

(c) detecting a mutation of at least one base in the coding region of a glaucoma-related gene; and

(d) predicting future onset of primary open angle glaucoma and/or normal ocular tension glaucoma using the mutation as an index.

11. An assaying reagent or an assaying reagent kit comprising an oligonucleotide of item 9.

### BRIEF DESCRIPTION OF THE DRAWINGS

FIG. 1 shows the structure of the OPTN gene (Example 1);

FIG. 2 shows the structure of exon 4 of the OPTN gene and the relation of positions between it and its corresponding primer (Example 1);

FIG. 3 shows the structure of exon 5 of the OPTN gene and the relation of positions between it and its corresponding primer (Example 1);

FIG. 4 shows the structure of exon 6 of the OPTN gene and the relation of positions between it and its corresponding primer (Example 1);

FIG. 5 shows the structure of exon 7 of the OPTN gene and the relation of positions between it and its corresponding primer (Example 1);

FIG. 6 shows the structure of exon 8 of the OPTN gene and the relation of positions between it and its corresponding primer (Example 1);

FIG. 7 shows the structure of exon 9 of the OPTN gene and the relation of positions between it and its corresponding primer (Example 1);

FIG. 8 shows the structure of exon 10 of the OPTN gene and the relation of positions between it and its corresponding primer (Example 1);

FIG. 9 shows the structure of exon 11 of the OPTN gene and the relation of positions between it and its corresponding primer (Example 1);

FIG. 10 shows the structure of exon 12 of the OPTN gene and the relation of positions between it and its corresponding primer (Example 1);

FIG. 11 shows the structure of exon 13 of the OPTN gene and the relation of positions between it and its corresponding primer (Example 1);

FIG. 12 shows the structure of exon 14 of the OPTN gene and the relation of positions between it and its corresponding primer (Example 1);

FIG. 13 shows the structure of exon 15 of the OPTN gene and the relation of positions between it and its corresponding primer (Example 1); and

FIG. 14 shows the structure of exon 16 of the OPTN gene and the relation of positions between it and its corresponding primer (Example 1).

### DESCRIPTION OF THE PREFERRED EMBODIMENTS

The inventors determined the coding sequence in the coding region of each exon, constituting a glaucoma-related gene, consisting of the base sequence denoted by SEQ ID NO: 1. In the course of this determination, it was verified that genetic polymorphism occurs with a difference in frequency in the coding region of the gene between the glaucoma patient group and the non-patient group. Furthermore, the onset rate of glaucoma depends thereon statistically in a significant manner compared to the rate thereof in the general group. The invention is constituted based on the above new findings.

[Glaucoma-Related Genes]

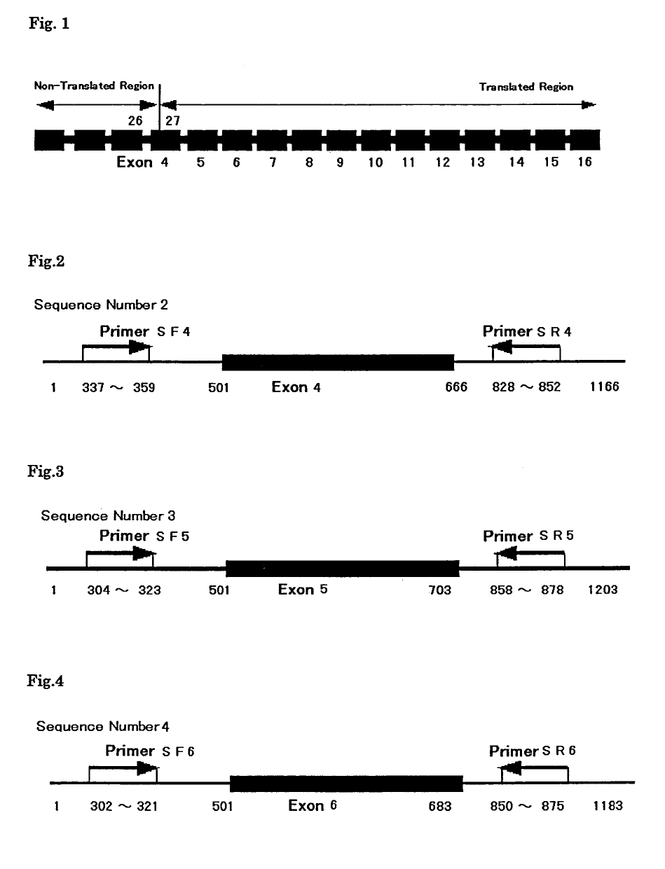

As an example of the glaucoma-related genes, OPTN (optineurin) gene can be noted. The structure and the coding sequence of the optineurin gene are as shown in FIG. 1 and as listed in Table 1 and, for example, there are coding regions to be transcribed and translated into protein, and non-translated regions as well as other elements. The base positions of the OPTN gene conform with the base numbers defined in SEQ ID NO: 1 (Genbank entry number AF420371, AF420372 and AF420373). The OPTN protein contains 13 exons. The sequence of each exon and the location of it is denoted by SEQ ID NO: 2 for exon 4, by SEQ ID NO: 3 for exon 5, by SEQ ID NO: 4 for exon 6, by SEQ ID NO: 5 for exon 7, by SEQ ID NO: 6 for exon 8, by SEQ ID NO: 7 for exon 9, by SEQ ID NO: 8 for exon 10, by SEQ ID NO: 9 for exon 11, by SEQ ID NO: 10 for exon 12, by SEQ ID NO: 11 for exon 13, by SEQ ID NO: 12 for exon 14, by SEQ ID NO: 13 for exon 15 and by SEQ ID NO: 14 for exon 16. The correspondence between the base sequence of the OPTN gene represented as the sequence denoted by SEQ ID NO: 1 and the base sequence of each exon represented as the sequences denoted by SEQ ID NOS: 2 to 14 is as follows. The positions 501-666 in SEQ ID NO: 2 to the positions 1-166 of SEQ ID NO: 1, the positions 501-703 in SEQ ID NO: 3 to the positions 167-369 of SEQ ID NO: 1, the positions 501-683 in SEQ ID NO: 4 to the positions 370-552 of SEQ ID NO: 1, the positions 501-574 in SEQ ID NO: 5 to the positions 553-626 of SEQ ID NO: 1, the positions 501-653 in SEQ ID NO: 6 to the positions 627-779 of SEQ ID NO: 1, the positions 501-603 in SEQ ID NO: 7 to the positions 780-882 of SEQ ID NO: 1, the positions 501-616 in SEQ ID NO: 8 to the positions 883-998 of SEQ ID NO: 1, the positions 501-650 in SEQ ID NO: 9 to the positions 999-1148 of SEQ ID NO: 1, the positions 501-594 in SEQ ID NO: 10 to the positions 1149-1242 of SEQ ID NO: 1, the positions 501-659 in SEQ ID NO: 11 to the positions 1243-1401 of SEQ ID NO: 1, the positions 501-631 in SEQ ID NO: 12 to the positions 1402-1532 of SEQ ID NO: 1, the positions 501-580 in SEQ ID NO: 13 to the positions 1533-1612 of SEQ ID NO: 1, the positions 501-622 in SEQ ID NO: 14 to the positions 1613-1734 of SEQ ID NO: 1.

[Gene Mutation]

A mutation in the glaucoma-related gene for detection in the present invention refers to substitution, in deletion and/or insertion of at least one base at a specific position(s) in the base sequence of the OPTN gene. The “specific position” refers to a position selected from the positions 619 and/or 898 of the base sequence denoted by SEQ ID NO: 1.

As the detailed substitution of bases at specific positions in the invention, a substitution of G for A at the position 619 and/or a substitution of A for G at the position 898 in the base sequence denoted by SEQ ID NO: 1 are mentioned.

[Assay Method]

In the method for assaying for the mutation of the glaucoma-related gene, the method is not limited, as far as detecting the specific mutation of the OPTN gene disclosed herein, but various methods currently known or to be known in the future can be used for detection of a relevant mutation.

In order to check for a mutation as disclosed in this invention in the OPTN gene of a subject, various methods can be used for analyzing the base sequences containing the position(s) of a possible mutation. As these methods, for example, Southern hybridization method, dot hybridization method (see J. Mol. Biol, 98: 503-517 (1975) etc.), dideoxy base sequence determination method (Sanger's method), the various detecting methods combined with DNA amplification approaches can be listed [for example, PCR-restriction fragments length polymorphism analyzing method (RFLP), PCR-single-chain-high-order structure polymorphism analyzing method (see Proc. Natl. Acad. Sci., U.S.A., 86: 2766-2770 (1989) etc.), PCR-specific sequence oligonucleotide method (SSO), allele specific oligonucleotide method using PCR-SSO and dot hybridization method (see Nature, 324: 163-166 (1986) etc.)] can be employed. For the position(s) of the gene mutation to be detected as disclosed and identified in this invention, those skilled in the art can detect the mutation using known methods.

[Preparation of the Sample for Measurement]

In order to analyze the OPTN gene of a subject, the sample to be prepared for the assay method of the invention is not specifically limited but may be any biological sample containing the OPTN gene of the subject, such as tissues collected from a living body, tissue cut out in an operation and tissue from the oral cavity mucous membrane, blood, serum, excretions, ejaculated semen, expectorated sputum, saliva, brain and spinal fluid, hair etc. For example, the OPTN gene extracted by a known gene extraction method such as phenol-chloroform method, from a biological sample such as tissue that has been crushed using a blender can be used as a sample to be examined. Furthermore, the extracted OPTN gene can be prepared as a sample to be examined by amplifying and concentrating it.

The sample to be examined may either be the full-length of the DNA of the OPTN gene or a DNA fragment (a portion of the full-length DNA). When a DNA fragment is examined, it is preferred for the fragment to include the complete or partial coding region of at least one (1), preferably, two (2) or more, and, more preferably, three (3) or more exons. There is no limitation on the DNA fragment in terms of its base length as long as it is useable for the detection of a gene mutation, i.e., as long as it has a length available for measurement as a sample DNA prepared for the examination of a base mutation. As the base length of such DNA, a length of around ten (10) or more bases and, preferably, around 20 or more bases can be selected. Generally, a length of around 100-1000 bases and, preferably, around 200-300 bases is selected.

Furthermore, the sample to be examined may either be DNA or a DNA transcription product. More specifically, the sample may either be a messenger RNA (mRNA) transcribed from DNA, cDNA further reverse-transcribed from the mRNA or other complementary DNA. All of the various steps that may be employed in the detection method for the gene mutation of the invention, such as, for example, synthesis of DNA or DNA fragment; enzyme treatments with the purpose of cutting, deleting, adding or coupling DNA; isolation, purification, duplication and selection of DNA; and amplification of DNA fragment may be performed according to conventional methods (see Methods for Experiments in Molecular Genetics, Kyouritsu Shuppan Co., Ltd., 1983, etc.). These steps can be modified according to conventional procedure as necessary.

Amplification of nucleic acid in order to prepare a sample to be examined may be implemented according to, for example, a PCR method or its variant method (see PCR Technology, Takara Shuzo Co., Ltd., 1990, etc.). In this case, an oligonucleotide capable of specifically forming a hybrid at a portion of a glaucoma-related gene, more specifically, an oligonucleotide having a primer function, is suitably selected so that it specifically amplifies a desired DNA fragment having at least one (1) or more of the above specific positions, related to mutation.

[Oliginucleotide Having a Primer Function]

As oligonucleotides having a primer function, an oligonucleotide such as, for example, (1) an oligonucleotide comprising a base sequence denoted by any of SEQ ID NO:s 15-40, (2) a complementary chain of an oligonucleotide described in above (1), (3) an oligonucleotide capable of hybridizing under stringent conditions with an oligonucleotide described in above (1) or (2), (4) an oligonucleotide having a homology of 60% or more to an oligonucleotide described in any of above (1) to (3), (5) an oligonucleotide including a base sequence in which one (1) to several bases are mutated by substitution, deletion, insertion or addition in an oligonucleotide described in above (1) to (4), and etc. can be used.

An oligonucleotide itself can be designed by known methods, i.e., for example, it can be chemically synthesized. Also, it is possible to cut natural chain(s) of nucleic acid using restriction enzyme etc., to modify the natural nucleic acid chain or to couple or cut chains so that the natural nucleic acid is constituted by an above base sequence. More specifically, oligonucleotides can be synthesized using an oligonucleotide synthesizing apparatus (manufactured by Applied Biosystems, Expedite Model 8909 DNA Synthesizer) etc. Furthermore, known production methods can be used as synthesizing methods of oligonucleotides having one (1) to several bases varied such as by substitution, deletion, insertion or addition. The synthesis can be carried out using, for example, portion-specific mutation introduction method, gene homologous recombination method, primer elongation method and polymerase chain reaction method (PCR) or using a plurality of these methods in combination. Furthermore, the synthesis can be carried out according to the methods described in, for example, Molecular Cloning; A Laboratory Manual, Second Edition, Edited by Sambrook et al., Cold Spring Harbor Laboratory Press, Cold Spring Harbor, N.Y., 1989; Labo Manual Genetic Engineering, Edited by Masami Matsumura, Maruzen Co., Ltd., 1988; PCR Technology, The Principle and Application of DNA Amplification, Edited by Ehrlich, HE., Stockton Press, 1989, etc. Furthermore, a modified version of an above method such as, for example, a technique by Ulmer (Science (1983) 219:666) can be used.

Generally known conditions can be selected as the stringent conditions for hybridization and, as an example thereof, after hybridizing over night at 42° C. in a solution containing 50%-formamide, 5×SSC (150 mM of NaCl, 15 mM of trisodium citrate), 50 mM of sodium phosphate (pH 7.6), 5× Denhart's solution, 10%-dextran sulfuric acid, and 20 μg/ml of DNA, a primary washing in 2×SSC·0.1% of SDS at room temperature and a secondary washing in 0.1×SSC·0.1% of SDS.

[Detection of a Mutation of Gene DNA]

A mutation of DNA can be detected by, for example, determining the base sequence of the OPTN gene contained in the sample to be examined using Sanger's method.

Using a one-chain target OPTN gene hybridized with a complementary oligonucleotide as a primer, a complementary chain is synthesized with DNA polymerase in the direction from the position 5′ to the position 3′. The oligonucleotide used as a primer is, for example, an oligonucleotide as described above.

A complementary chain is synthesized by adding a small amount of dideoxyribonucleotide-triphosphoric acid (ddNTP) to each base in addition to four (4) kinds of deoxyribonucleotide-triphosphoric acid (dNTP) as substrates for the reaction. ddNTP is an analogue (analogous substance) having an —H group at the position 3′ of a deoxyribose instead of an —OH group. Once ddNTP is taken in instead of dNTP, the complementary chain is not synthesized further and DNAs having various lengths are obtained. In the reaction system, the synthesized DNA can be labeled by adding, for example, a primer or dNTP labeled with a chemiluminescent substance or a radioactive isotope. Then, the base sequence can be determined by the electrophoresis of reaction products with denatured polyacrylamide.

For example, Klenow enzyme, T7 phage and thermophilic-bacteria-originated DNA polymerases can be listed as DNA polymerase used in Sanger's method. The exonuclease activity of these DNA polymerases is commonly removed in a genetic-engineering approach. At first, the target genes were used in a form of one chain in Sanger's method but, at present, a method is often used in which double-chain plasmid is alkali-denatured as it is and is used in the detection method.

Sequence reactions can be carried out by Sanger's method or a cycle sequence method. Cycle sequence method is a method formed by combining Sanger's method and the PCR method. In this method, template DNA does not need to be single-chain DNA. The reaction is carried out with DNA, one kind of primer, dNTPs, ddNTPs and heat-resistant DNA polymerase in the reaction system. It is the same as Sanger's method in that, during the PCR reaction, ddNTPs are taken up, elongation is terminated and, as a result, DNA having the same bases at the 3′-terminal position is synthesized. As sequence reactions for automatic sequencers, there are dye primer method in which the primer is marked with fluophor, dye terminator method in which ddNTP is marked with fluophor, internal-label method in which dNTP substrate is labeled etc.

[Assaying Reagent and Assaying Reagent Kit]

The invention also includes an assaying reagent and an assaying reagent kit used for the gene assay method for predicting glaucoma. The assaying reagent may be any of the various reagents used for the method of the invention such as, for example, a primer for amplification of the sample to be examined, a primer for determining the base sequences of the sample to be examined, various polymerase, base substrates, marking materials, etc. The assaying reagent kit may be any kit in which at least two (2) of the reagents used for the method of the invention are present.

### EXAMPLES

The invention will be specifically described referring to examples. However, the invention is not limited to those examples.

### Example 1

DNA Analysis of OPTN Gene

(1) Extraction of DNA

Blood provided by a subject was processed according to a conventional method and DNA was extracted from an eukaryotic cell. Using a kit product named “Dr. GenTLE™ (for blood) (manufactured by Takara Shuzo Co., Ltd.,)” as the DNA extraction kit, DNA was extracted according to the protocol provided by the instruction manual of the product.

(2) Amplification of Template DNA

Using the obtained DNA extraction solution as a template, OPTN gene was amplified in PCR using a kit product named “LATaq (manufactured by Applied Biosystems)”, a kit for PCR amplification.

The sequence of each exon from exon 4 to exon 16 is disclosed in a Genbank entry No. NT_{—}031849 and each sequence is represented by the base sequence of the region described as follows in the sequences denoted by SEQ ID NO:s 2 to 14

Exon 4: SEQ ID NO: 2, the positions 501-666

Exon 5: SEQ ID NO: 3, the positions 501-703

Exon 6: SEQ ID NO: 4, the positions 501-683

Exon 7: SEQ ID NO: 5, the positions 501-574

Exon 8: SEQ ID NO: 6, the positions 501-653

Exon 9: SEQ ID NO: 7, the positions 501-603

Exon 10: SEQ ID NO: 8, the positions 501-616

Exon 11: SEQ ID NO: 9, the positions 501-650

Exon 12: SEQ ID NO: 10, the positions 501-594

Exon 13: SEQ ID NO: 11, the positions 501-659

Exon 14: SEQ ID NO: 12, the positions 501-631

Exon 15: SEQ ID NO: 13, the positions 501-580

Exon 16: SEQ ID NO: 14, the positions 501-622

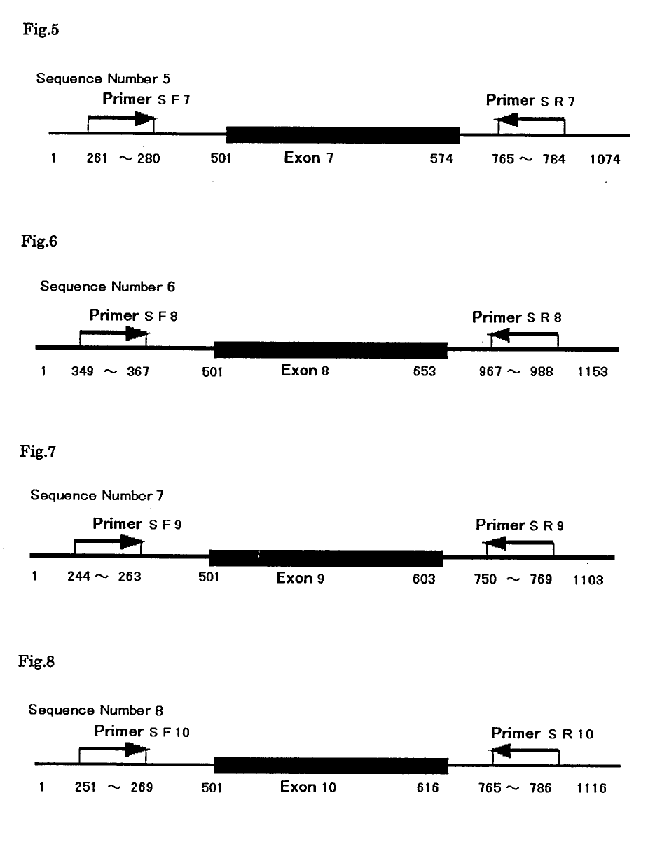

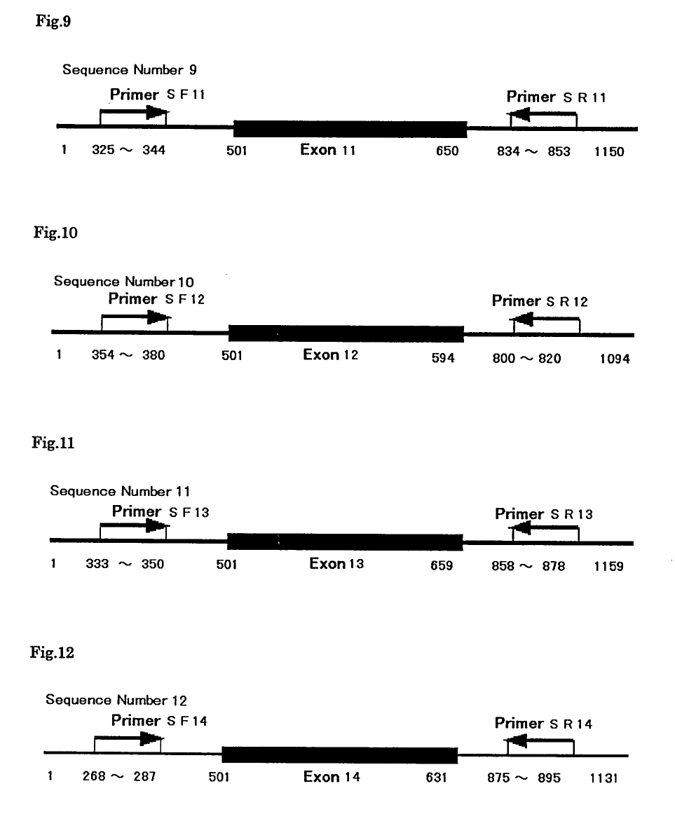

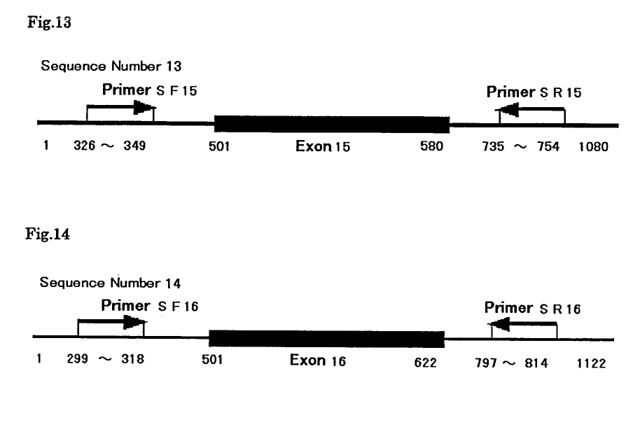

Oligonucleotide represented by each of the base sequences denoted by SEQ ID NOS: 15 to 40 was used to amplify each exon. The relation of positions between each primer and each exon is shown in FIGS. 2 to 14. However, each antisense primer consists of sequences based on a complementary chain of the sequences denoted by SEQ ID NOS: 2 to 14.

Primer for Exon 4

Normal Chain, SF4, SEQ ID NO: 15; region=SEQ ID NO: 2, the positions 337-359

Inverted Chain, SR4, SEQ ID NO: 16; region=the complementary sequence of SEQ ID NO: 2, positions 828-852

Primer for Exon 5

Normal Chain, SF5, SEQ ID NO: 17; region=SEQ ID NO: 3, the positions 304-323

Inverted Chain, SR5, SEQ ID NO: 18; region=the complementary sequence of SEQ ID NO: 3, positions 858-878

Primer for Exon 6

Normal Chain, SF6, SEQ ID NO: 19; region=SEQ ID NO: 4, the positions 302-321

Inverted Chain, SR6, SEQ ID NO: 20; region=the complementary sequence of SEQ ID NO: 4, positions 850-875

Primer for Exon 7

Normal Chain, SF7, SEQ ID NO: 21; region=SEQ ID NO: 5, the positions 261-280

Inverted Chain, SR7, SEQ ID NO: 22; region=the complementary sequence of SEQ ID NO: 5, positions 765-784

Primer for Exon 8

Normal Chain, SF8, SEQ ID NO: 23; region=SEQ ID NO: 6, the positions 349-367

Inverted Chain, SR8, SEQ ID NO: 24; region=the complementary sequence of SEQ ID NO: 6, positions 967-988

Primer for Exon 9

Normal Chain, SF9, SEQ ID NO: 25; region=SEQ ID NO: 7, the positions 244-263

Inverted Chain, SR9, SEQ ID NO: 26; region=the complementary sequence of SEQ ID NO: 7, positions 750-769

Primer for Exon 10

Normal Chain, SF10, SEQ ID NO: 27; region=SEQ ID NO: 8, the positions 251-269

Inverted Chain, SR10, SEQ ID NO: 28; region=the complementary sequence of SEQ ID NO: 8, positions 765-786

Primer for Exon 11

Normal Chain, SF 1, SEQ ID NO: 29; region=SEQ ID NO: 9, the positions 325-344

Inverted Chain, SRI 1. SEQ ID NO: 30; region=the complementary sequence of SEQ ID NO: 9, positions 834-853

Primer for Exon 12

Normal Chain, SF12, SEQ ID NO: 31; region=SEQ ID NO: 10, the positions 354-380

Inverted Chain, SR12, SEQ ID NO: 32; region=the complementary sequence of SEQ ID NO: 10, positions 800-820

Primer for Exon 13

Normal Chain, SF13, SEQ ID NO: 33; region=SEQ ID NO: 11, the positions 333-350

Inverted Chain, SR13, SEQ ID NO: 34; region=the complementary sequence of SEQ ID NO: 11, positions 858-878

Primer for Exon 14

Normal Chain, SF14, SEQ ID NO: 35; region=SEQ ID NO: 12, the positions 268-287

Inverted Chain, SR14, SEQ ID NO: 36; region=the complementary sequence of SEQ ID NO: 12, positions 875-895

Primer for Exon 15

Normal Chain, SF15, SEQ ID NO: 37; region=SEQ ID NO: 13, the positions 326-349

Inverted Chain, SRI 5, SEQ ID NO: 38; region=the complementary sequence of SEQ ID NO: 13, positions 735-754

Primer for Exon 16

Normal Chain, SF16, SEQ ID NO: 39; region=SEQ ID NO: 14, the positions 299-318

Inverted Chain, SR16, SEQ ID NO: 40; region=the complementary sequence of SEQ ID NO: 14, positions 797-814

The PCR reaction was carried out such that a cycle of heating 30 seconds at 94° C., 30 seconds at 60° C. and 30 seconds at 72° C. was repeated for 30 times.

(3) Determination of the Base Sequence of DNA fragment of Each Exon

The base sequence of the DNA fragment obtained by above PCR for each exon was determined using an automatic DNA sequencer ABI Prism3100 (manufactured by Applied Biosystems) according to the protocol provided by the instruction manual of the sequencer. In this procedure, the cyclic sequence reactions were carried out with either a forward primer or a reverse primer among the primers used in PCR reactions for each exon. (4) Determination of the Base Sequence of DNA fragment of Each Exon in the Control Group.

Furthermore, blood obtained from a group of non-patient volunteers as a control group was processed according to the above approach and the base sequence dominant in the non-patient group was determined for DNA fragment of each exon.

### Example 2

(1) Analysis of Polymorphism of the Base Sequence of OPTN Gene

Blood obtained from patients diagnosed to have open angle glaucoma by a medical institution was processed according to the approach used in the above Example. Then, the base sequence of the OPTN gene contained therein was studied and the sequence was compared to base sequences of the non-patient group.

Table 1 shows the result of the above. The first line in Table 1 lists exon numbers, the second line lists the SEQ ID NO:s representing each exon, the third line lists the base positions in each sequence and the fourth line lists the change of bases detected as a mutation.

As a result, a mutation was recognized only for the patient group and no mutation was recognized for the non-patient group at the base position 567 in the SEQ ID NO: 5, i.e., the base position 619 in the SEQ ID NO: 1 and at the base position 516 in the SEQ ID NO: 8, i.e., the base position 898 in the SEQ ID NO: 1.

TABLE 1

| | | |
|---|---|---|
| Exon | 7 | 10 |
| SEQ ID NO: | 5 | 8 |
| Base Position | 567 | 516 |
| Position in the | 619 | 898 |
| SEQ ID NO: 1 | | |
| A mutation | A to G | G to A |
| Freguency in a patient group | 1.4% | 0.8% |
| Frequency in a non-patient | 0.0% | 0.0% |
| group | | |

As has been set forth, the disclosure obtained concerning gene mutation according to the invention is effective for predicting future onset of glaucoma. When the future onset of, especially, open angle glaucoma can be predicted by detecting a mutation of the OPTN gene by a gene assay method of the invention, it is possible to prevent the onset of the disease or to treat the disease early on.

What is claimed is:

1. A gene assay method comprising the steps of:
detecting a mutation of at least one base in the coding region of an optineurin(OPTN) gene of a human subject; and predicting future onset of glaucoma in the subject using the mutation as an index.

2. The gene assay method of claim 1, wherein the coding region of said glaucoma-related gene is an OPTN gene has a nucleic acid sequence denoted by SEQ ID NO: 1.

3. The gene assay method of claim 2, wherein said mutation is a substitution of G for A at position 619 and/or a substitution of A for G at position 898 in the nucleic acid sequence denoted by SEQ ID NO: 1.

4. The gene assay method of claim 2, wherein said mutation is a deletion of one or more bases in the nucleic acid sequence denoted by SEQ ID NO: 1.

5. The gene assay method of claim 2, wherein said mutation is an insertion of one or more bases in the nucleic acid sequence denoted by SEQ ID NO: 1.

6. The gene assay method of claim 2, wherein said mutation is two or more substitutions of bases in the nucleic acid sequence denoted by SEQ ID NO: 1.

7. The gene assay method according to claim 1, wherein the glaucoma is primary open angle glaucoma and/or normal ocular tension glaucoma.

8. The gene assay method according to claim 1, wherein the mutation is detected by using an oligonucleotide capable of forming a hybrid at a specific position of the coding region of the OPTN gene.

9. An oligonucleotide selected from the group consisting of oligonucleotides comprising sequences as follows:
(1) an oligonucleotide consisting of a base sequence represented by any of SEQ ID NOs: 15 to 40;
(2) a complementary chain of an oligonucleotide according to (1);
(3) an oligonucleotide that hybridizes with an oligonucleotide according to (1) or (2) under stringent conditions;
(4) an oligonucleotide having a homology of 60% or more to an oligonucleotide according to any one of (1) to (3); and
(5) an oligonucleotide according to any one of (1) to (4) having one to several bases mutated by substitution, deletion, insertion or addition.

10. A gene assay method for predicting future onset of primary open angle glaucoma and/or normal ocular tension glaucoma, comprising the steps of:
(a) isolating a polynucleotide sample from a subject suspected of having a mutation in a glaucoma-related gene,
(b) performing a nucleic acid amplification process on said polynucleotide using at least one oligonucleotide selected from the group consisting of oligonucleotides comprising sequences as follows:
(1) an oligonucleotide consisting of a base sequence represented by any of SEQ ID NOs: 15 to 40;
(2) a complementary chain of an oligonucleotide according to (1);
(3) an oligonucleotide that hybridizes with an oligonucleotide according to (1) or (2) under stringent conditions;
(4) an oligonucleotide having a homology of 60% or more to an oligonucleotide according to any one of (1) to (3); and
(5) an oligonucleotide according to any one of (1) to (4) having one to several bases mutated by substitution, deletion, insertion or addition
(c) detecting a mutation of at least one base in the coding region of a glaucoma-related gene; and
(d) predicting future onset of primary open angle glaucoma and/or normal ocular tension glaucoma using the mutation as an index.

11. An assaying reagent or an assaying reagent kit comprising an oligonucleotide of claim 9.


SEQUENCE LISTING (40 sequences)

SEQ ID NO: 1 - DNA, 1734 bp, Homo sapiens
atgtcccatcaacctctcagctgcctcactgaaaaggaggacagccccagtgaaagcacaggaaatggacccccccacctggcccacccaaacctggacacgtttaccccggaggagctgctgcagcagatgaaagagctcctgaccgagaaccaccagctgaaagaagccatgaagctaaataatcaagccatgaaagggagatttgaggagctttcggcctggacagagaaacagaaggaagaacgccagttttttgagatacagagcaaagaagcaaaagagcgtctaatggccttgagtcatgagaatgagaaattgaaggaagagcttggaaaactaaaagggaaatcagaaaggtcatctgaggaccccactgatgactccaggcttcccagggccgaagcggagcaggaaaaggaccagctcaggacccaggtggtgaggctacaagcagagaaggcagacctgttgggcatcgtgtctgaactgcagctcaagctgaactccagcggctcctcagaagattcctttgttgaaattaggatggctgaaggagaagcagaagggtcagtaaaagaaatcaagcatagtcctgggcccacgagaacagtctccactggcacggcattgtctaaatataggagcagatctgcagatggggccaagaattacttcgaacatgaggagttaactgtgagccagctcctgctgtgcctaagggaagggaatcagaaggtggagagacttgaagttgcactcaaggaggccaaagaaagagtttcagattttgaaaagaaaacaagtaatcgttctgagattgaaacccagacagaggggagcacagagaaagagaatgatgaagagaaaggcccggagactgttggaagcgaagtggaagcactgaacctccaggtgacatctctgtttaaggagcttcaagaggctcatacaaaactcagcaaagctgagctaatgaagaagagacttcaagaaaagtgtcaggcccttgaaaggaaaaattctgcaattccatcagagttgaatgaaaagcaagagcttgtttatactaacaaaaagttagagctacaagtggaaagcatgctatcagaaatcaaaatggaacaggctaaaacagaggatgaaaagtccaaattaactgtgctacagatgacacacaacaagcttcttcaagaacataataatgcattgaaaacaattgaggaactaacaagaaaagagtcagaaaaagtggacagggcagtgctgaaggaactgagtgaaaaactggaactggcagagaaggctctggcttccaaacagctgcaaatggatgaaatgaagcaaaccattgccaagcaggaagaggacctggaaaccatgaccatcctcagggctcagatggaagtttactgttctgattttcatgctgaaagagcagcgagagagaaaattcatgaggaaaaggagcaactggcattgcagctggcagttctgctgaaagagaatgatgctttcgaagacggaggcaggcagtccttgatggagatgcagagtcgtcatggggcgagaacaagtgactctgaccagcaggcttaccttgttcaaagaggagctgaggacagggactggcggcaacagcggaatattccgattcattcctgccccaagtgtggagaggttctgcctgacatagacacgttacagattcacgtgatggattgcatcatttaa

SEQ ID NO: 2 - DNA, 1166 bp, Homo sapiens
tgcaagctctgcctcccgggttcacgccattctcctgcctcagcctcccgagtagctgggactacaagcgcccaacaccaagcccggctaattttttgtatttttagtagagacggggtttcactgtgttagccaggatggtctcaatctcctgacctcatgatctgtccgcctcggcctcccaaagtgctgggattacaggcgtgagccaccacgcccggccctcattgtacccttttatacacccatacacacacacgcacacacacacatgcacacatgcgcgtgcacacacacacacacttttctgaagctacatataccttttttgtttaaaaggaagaatcaaaaatgtccaaaatgtaactggagagaaagtgggcaacttttggagtaagtattagcaatcgccaatgggtttgtgggactcccggggaccccttgtggggcgggggacagctctattttcaacaggtgacttttccacaggaacttctgcaatgtcccatcaacctctcagctgcctcactgaaaaggaggacagccccagtgaaagcacaggaaatggacccccccacctggcccacccaaacctggacacgtttaccccggaggagctgctgcagcagatgaaagagctcctgaccgagaaccaccagctgaaaggtgagcagggctggcccctgtgtgccccattcatcctgggcctgcaagaaatgccatccctttgcactaaggcttggtggtgagctcccttctccccgtttccataggtggtagctggtggggaagcacaggatttagcatttggcaaggctaaatctgttctgatttttacttttggaaacaggtacaagtaaaaactgtgtgtatctcaaggaagtagcataatgatatttagcccattcaaaaggaaaaagaggctgggcgtggtggctcatgcctgtcattccatcactttgggaggccgaggcagaaggattgcttgagtacaggagttcaagaccagcctgggcaagatggcaagacctgatctctacaaaaaaattaaaaaaaaaaaaaaaaagctgggcgtggtggtgcacgcctctggtcctagctactggggatgctgaggttggaggattgcttgagcctgggaagttggagctgcagtgagccatgat

SEQ ID NO: 3 - DNA, 1203 bp, Homo sapiens
gcagtgagccatgatcgtgccactgcactttagcctggatgacagagagagaccctgactcaaaaaaaaaaaaaaaaaaaggaaaaaggaagaaaggctgctatggttccagagttagtcctatatattaccttattaagagaaagcatcctggtatctcaagatggctttgggcaggaccagtatttgaatctaggagtagtaagaacttccttagctcctagtaaccatagatatttagatatttgtgctgtagtggcggtacccaaatccactttattttcttgggatttttaaggactagaaatgatgttcatcccgctagtcttttctgtaagcaaaaaccacttcgtctttttgctgctgacccttgggccaaggctaagcatggcatctttcaattcagagccatgtggtcaagtggactagagggagatttggttcatcagatcaagtccactttcctggtgtgtgactccatcactctgaacctcctgcagaagccatgaagctaaataatcaagccatgaaagggagatttgaggagctttcggcctggacagagaaacagaaggaagaacgccagttttttgagatacagagcaaagaagcaaaagagcgtctaatggccttgagtcatgagaatgagaaattgaaggaagagcttggaaaactaaaagggaaatcagaaaggtcatctgaggtgagcagaccgatccattgtgatgttgttttttttttttcccttgacatttgcagtggaatcttacgtgtctagactcctagatcaaaacctttcatggttcagtctggattggtgttttgcctggtcttggaagaagtgcttttgctgaaaagattggttgccctattaagggtcatggataatctcttttagaagaaagaaatttgtaaagctttgaccgtactgattgtaggcaaaagaacagtaaggttataaatcattgtattgtattcattatagatggtgcagatgggcctctgcctagaaccaacaattgtttttagtttgtctttgatataaaaaatatgtttaaaaaacccattactcagaatttttacttgttgaccttgtctgttctctcagtctaaaatggagattattcactttacattttcctttttaaaaatgctttggaaaatgtcatgttgtggtaggaggctatcgcattgccacagatg

SEQ ID NO: 4 - DNA, 1183 bp, Homo sapiens
ttgtcctgcctcagcctcccgagtagctgggactacaggcgcccgccaccacgcccagctaatctttttgtatttttagtagagacggggtttcactgtgttagccaggatggtctccatctcctgaccttcatgatccgcccacctcggcctcccaaagtgctgggattacaggcgtgagccaccacgcctggcttggcttttttttttttttttttgagacagggtcttggcagtcttaaactcctgggctcaggcagtcttcctgcctcagcctcccaactaatggggactacaggtgtgtgccactacacctggctaattattaaattttttgtaaagatgggggtcttgctatgttgcccaggctggtctcaaaatcctggcctcaagggatcctcccacttcagcctcccagagctctgcgattaagggcatgagcccatggtgcccagccttagtttgatctgttcattcactttactccttgtcatctccaggaccccactgatgactccaggcttcccagggccgaagcggagcaggaaaaggaccagctcaggacccaggtggtgaggctacaagcagagaaggcagacctgttgggcatcgtgtctgaactgcagctcaagctgaactccagcggctcctcagaagattcctttgttgaaattaggatggctgtgagtttttggttttatttttgttttgagcaaactataaagcctcccctggaaagatgaaacaaataccactttttcttgtcaacacaagccaaggattgaggaaattccagtgtagcaaagataaattggctctcattttctaagtatagcataatgcatgtaagggttatcatagctaaaatggaaaaatattaattaccttttatgatgaaagctgtagtctttttttttttcttcatcatgtcctggcaaattgaacatttttgtgaccagaaaaggaaaaaacccacacgaacatgaactttctgtcatttttcaaactaggtctcaaagctgtattccgcagttcacttaagggagcgcaaacatattttcacaacagaaccctctttttttgttttgagacagagtcttactctgtcttcccggctggaatgcagtgatgtgatctcggctcactgcaccctctgcctccggggttcaagagattctcgtgc

SEQ ID NO: 5 - DNA, 1074 bp, Homo sapiens
agtgacctgtggtgcatacaaatttctaatgggaaccaacttggccaagatggtgctttgtgaatctcattcacagaaactgcctcttttttaactttacctcagtgagttctagcattttgcattttaaaggaaggatatgtggagttgtcaccagctctgtatgaccttaaccttgagaaagagggaactgccaaggaaagggaggagcagataagctttcatgtttacagagtcaggtagaatgtgtatggcgagatgaaactgaccttcacgccttagctgggatatttataatcccgacagggcgtgccaggtgaggggagggtacgtttccatttcctctgagccaccccgtttaaacagtgcacatctgaatgtttggaagcttccttgggttgcatgtcacaaaaattcatcttttgtctttttcttcttttgacaaagaatttgtcttgtagacatattgtgttaaatcccttgcatttctgttttcacaggaaggagaagcagaagggtcagtaaaagaaatcaagcatagtcctgggcccacgagaacagtctccactggcacgtatgtgaaggaagactcgggctgtcaggcagacaggctgggcaggctcgtcactgggtgcttgtcaccggaggtcaaatgttgtgacctgaggaagtaacttctttatgatttataccaggatctttccagaatatttggtttgaatgctatttaatgttgcagctcaaactggcaaagattaaaaactgtttggttcctgtttggctcacactgactgctctgttctagtggtgtctcacctccagcagatgaaaagtgaaagcaaactggttctcaatcaagtcaatgatttgttcctaatcaaagacatgtttgctcattggttccccggtgccatttgacccagaccagcctgcccagcttccataagtgaaatattttcattttcttttccctgctacttcccagttataagctggcatggccaatactggaacatcttttgtaacaatgactgatagcactctcagtcattgtgggtgttgcctgaaagtgcc

SEQ ID NO: 6 - DNA, 1153 bp, Homo sapiens
atttctctgctctcattatttgaaaccacaagtgaaaaaggttttctccccttgacttaagctgtgatggtctctgttaacttggagaaaggccagtggtctgtacaatgtgcctttatcttttgtctgactgcagtcccctttgagactagatctctggaaagcttggcaccttcagccacggctgcctctgctgaactgttccgtgagttttgtggtgtggtgtgaggtacacagtgactgtttggaggacgtgggtgtgtgcattgtaagctggcctctccagagcctcactgagtctccacaccttccctaggaagcatggaggagcttggcactgggggtcccaggaccagctgtgcttgttcactagttgagaattagttggagaatgttctggaaagcagttcctttaagctggtcccagttatattgggttactctcttcttagtctttggaatttttctgatgaaaaccttttaacctttatactgaacagggcattgtctaaatataggagcagatctgcagatggggccaagaattacttcgaacatgaggagttaactgtgagccagctcctgctgtgcctaagggaagggaatcagaaggtggagagacttgaagttgcactcaaggaggccaaagaaaggtatgaaataggttaacttgaaatatgtgtttttttaaaacagctttcctgagatataattaagataccatacagttcacccatttaaagtatacatttcagtgttttttagaatattccaggattgtgcaaccactgttactacaatataattttagaacattttttcccccaaacagcactcactgtctgctcctccaagcaatgtgctttctgtctctatagatttggccattctagacatttcatataaatggaattatacagtctgtggttttttgtgactggcttctttcacgtagcataatgtttttgaggttcatctacaacgtagcatgtatcagtacttccttttccttgctgaataaccttccattgtctatatatacaacattttgtttattcattcatcagttgataaacattagagttgttgccactttttacctattaggaataatgctgctatgaacagtgtgtacaagtttttactgggatat

SEQ ID NO: 7 - DNA, 1103 bp, Homo sapiens
ccacagtctcttgtttcatttggattgggacggctttcctgtggttatgatttggtgttaagaatggtgttactttttttgttgtcgtttattcggtgacttttaaacttagctgtgtcctaaaaggaaaagtctttccttctctaatgaattcttatgaatgagataccatgttcatggaacacacatgcatccacatgtgtaaacacaaacaatttcaaaaacattgctgcataggacagttgcatggaaacaaatggtgttcaagatgagtttcacttgccttttacctctgtgtgtatttgtctgtgaatcaattctagccaattttaggatgaaaaataaaactaatgctaatatagtgaatgtgtagagattttgaaaacccctgatcctttatcccaattgtaaacaatgttctttttagtacttctgtaataattgctatttctcttaaagccaaagagaaagtaacttttctatcttctgtgattttccagagtttcagattttgaaaagaaaacaagtaatcgttctgagattgaaacccagacagaggggagcacagagaaagagaatgatgaagagaaaggcccggagactgtgagtcctaagattccacggccactaccacacccacacacacgagagtagtccagccactgaattcaaatcttgtgatgggttatttgctttagaaatatagaaatcatgttgatattgaatattatctatctattccttttatatgtccttgtcctgctctgtgtcaattgtagcgagatgtatttcttttttgttgttgttgttggagatggagtctcactctgtcgccaggctggagtgcagtggcacgatctcagctcactgcaacctccgcctcccaggttcaagcagttctcctgcctcagcctcccaagtagctgggattacaggtgcccgtcaccacgcctggctaatttttgtatttttaatacagacagggtttcaccatgttggccaggatggtcttgatctcttgacctcgtgatcctcccacctcggcctcccagagtgctgggattacagatatgagccactgcgcccagctgcaagatgtattt

SEQ ID NO: 8 - DNA, 1116 bp, Homo sapiens
acgaattcaacagccagtagcagggaaatatggtctttcaaggcatcagaaactcatttacaaaaattatagagctgccaggaaaaaggctgcacaacaaaaatagttgagtaaactagaaacatacactgggaagagagtatgggggcaagttgttagctggatagataggactgtgctttgacacctctgtggtctatgatctctgaacctggaatagggttcattttaatagcgataaagtcattatcccagtgcatccaaattgattagttcatgctttattaggaaacagaagttacccaaaacttagcaaacctaagtaccaagtatccaaaacattcttttcctacacaatgtttggggtattgtcaaagttggattgattcaccagccagtcttaattggctactaatggttcagcctgttttctcctaaagaggtttgtttaatgtcagatgataattgtacagatatgtttgggatttcccgtatgataggttggaagcgaagtggaagcactgaacctccaggtgacatctctgtttaaggagcttcaagaggctcatacaaaactcagcaaagctgagctaatgaagaagagacttcaagaaaagtaagaatgagagagcaattttatcctcctttgaaatatacatttttacaaagtatactactatataaaaacatagttttttaactatgttatgactaaaagaaaaatagacacctaattaaaatataaattcagaatatactaatgttccagttaatgtgtgagcatgaaatacttgtaagatggggggttggggactggagaactttaattctgccatttaggggcatttgttaaatgtacgagcctgggtaagatctctacagtaaagctgtgagctagttttcctgttactgacttaagctgatgacattgatgtgagtaagcataaagaaagatgaaaagagcataaagatcttgagtgacatttatttggaaaaaggtcaatttcaatttgttatttcaatcagttaattatttcaggctaacatgtagattgagcgtttggcatttgcttgtttctcttgatgtaagaagttacccaaaacttagcaaaccta

SEQ ID NO: 9 - DNA, 1150 bp, Homo sapiens
atgctttgtgcatagctgtcatttatttgtattatattgaaatcctctttccgatctttaagaagacttaggggaacttcctttttcccttattgaatctttgtcagaaactaaagtctttgcaattgacagaacctataactttttttttaatataaaagatatccacacatcactacatgagaagcgccttagctaattactactgtggtctgtgtttaaatactaaaaatgtatctgtatgactagtttaaacaattattcaaagaggacagtactgcatgtgagcttagatctgtacttttttatgtttaggcgtaagggttcagaaatatggccaggtctagtgaagaagcaaggaggattatgtatttcattttgcattcataaaccctacagccctaaaattcttatattgtacataaccttggggtttgtttaaaagccactgcgacgtaaaggagcattgtttatcctcatgaaatcttgacctttcttaggtgtcaggcccttgaaaggaaaaattctgcaattccatcagagttgaatgaaaagcaagagcttgtttatactaacaaaaagttagagctacaagtggaaagcatgctatcagaaatcaaaatggaacaggctaaaacagaggatgaaaagtgagtatgttgagtcagaagggcagcgacggggcagaggagggagaatcgcctttttatacagattggaattcggatttgagaataaattttaaaaaatttctttttcacttatctgaaggagtcctagcagacctctcagagagggggataaaatttaaaagttttgtcataataaaattatgctgattgtttgcactctgtcttgatttttcagaaaagattttttttgagagtaagaaatgctagtaggtcgtggggtgataaaggtaggcgagaagatttttctactggagtgttcagaaggttgggaggcaagactataagtttctatgatattttccccaggattccattttttaatatcttttttaataggtccaaattaactgtgctacagatgacacacaacaagcttcttcaagaacataataatgcattgaaaacaattgaggaactaacaagaaaagaggtattcactgaaaaaaattacttccata

SEQ ID NO: 10 - DNA, 1094 bp, Homo sapiens
gcaattccatcagagttgaatgaaaagcaagagcttgtttatactaacaaaaagttagagctacaagtggaaagcatgctatcagaaatcaaaatggaacaggctaaaacagaggatgaaaagtgagtatgttgagtcagaagggcagcgacggggcagaggagggagaatcgcctttttatacagattggaattcggatttgagaataaattttaaaaaatttctttttcacttatctgaaggagtcctagcagacctctcagagagggggataaaatttaaaagttttgtcataataaaattatgctgattgtttgcactctgtcttgatttttcagaaaagattttttttgagagtaagaaatgctagtaggtcgtggggtgataaaggtaggcgagaagatttttctactggagtgttcagaaggttgggaggcaagactataagtttctatgatattttccccaggattccattttttaatatcttttttaataggtccaaattaactgtgctacagatgacacacaacaagcttcttcaagaacataataatgcattgaaaacaattgaggaactaacaagaaaagaggtattcactgaaaaaaattacttccatagcctagtaatgaacagaaactgttgaacgttttgtatataaaatagttacatgaatccttcactaaatctggtttcaaaggttgttttccaatgtatcattatttcttgcatctagggtttgtaacttctgatgttccacatatgtgtaatgtgctttattgcgtacaaagatgatgtgaatgtcctatggtcagggattaagcacttcgtatttctttttttttttttttgagacggagtctcgctctgtcgcccaggctggagtgcagtggcgcgatctcggctcactgcaagctccgcctcctgggttcacgccattctcctgcctcagcctcccgagtagctgggactacaggcgcccgccaccgcgcccggctaattttttgtatttttagtagagacggggtttcaccttgttagccaggatggtctcgatctcctgacctcgtgatccacccgcctcggcctccc

SEQ ID NO: 11 - DNA, 1159 bp, Homo sapiens
gtgctgggattacaggtgtgagccatcatgcccagcagtagtgttcctctcttggacctaataattttaaatttaaaacatgtttcttcttttccactgactgcaggaagtaacaagtggcaaaataacagtatcaacgagtcacagccttattaacattggagtttgttattgtatccctgatttcggtgttatcaccttttttttaggaattcattatttgcaagccacaacttaaatacaactttctgaataagttagcgttgctgattaatagactggttagagctgatacattttttagatctcgctatgttgcccaggcttgtctcccactcctgggctcaaacgatcctcccacctcagcctctcaattctaggcatgagccaccacacccggccagagctgataattaaaaaaataaacctttttctaatattttactaaaacaggcagaattatttcaaaaccatttctagaataaatgtttctttttcagtcagaaaaagtggacagggcagtgctgaaggaactgagtgaaaaactggaactggcagagaaggctctggcttccaaacagctgcaaatggatgaaatgaagcaaaccattgccaagcaggaagaggacctggaaaccatgaccatcctcagggctcaggtgaggcaccttccaaaaccccagctgagcgaggccagccctgactgtattctcgcattggaaagcaatggtgtttagaatgtttgtaattttctattttatatattttttcacccgtgagtgtattaaaactttaaaattgaaacatttggaaagtgctcagtggatcttatctgttctacatttaataggtaattggattctttccagtttgtggcattatgattaacgttgctaagacattcctgtgcatgttgctctgttcacatgtggatattttatatttctgttgggtacacacctaggagtggagtcgctggatcataggctctgcatgttactcacttttaacaggtaatgccaaacagttttccagagtggttggaccagttttcactcccatcaacagagagtttccatggctctacatcttaccaacacttctattatcagtcattttcctttaaccactctggagggtatatagtggtatctcattt

SEQ ID NO: 12 - DNA, 1131 bp, Homo sapiens
tttcataaggtaaaataagatagtaaatgtaaagcacccaacataggacctcacacatgtttggaatttaacaaatagcatctatttgtgatgattattcttttaaatttagcttaagaccagccttcataaatacacctggcagaatcaatttactatattaagtaatcatttactatattaagttgatcctgaattgtttattatctaaaagtccagataattttgctgaattaatggtacctacagtatttaaactacctatatcagtgcagttgcaggatttgtgttgtttaaagcacacacacaaacacagcttgtatctgctatcggaatgtacctggaaagtcatggtcattatactgttttctagcaggattgtgcatctgtgattcacaagggctattgaaggatacagcactacctcctcatcgcataaacactgtaagaatctgcattcatctaggtactaacttctgtatctttttttcctctaacagatggaagtttactgttctgattttcatgctgaaagagcagcgagagagaaaattcatgaggaaaaggagcaactggcattgcagctggcagttctgctgaaagagaatgatgctttcgaagacggaggcaggtaaggaaaagagagaggaggacccagagctcacatcagcatggccgtagaagaggtgcctgtccaaagacgttcctgatttgaactataagaatagctgtgttcgcgccactgcactcctgcctaggtgacagagcgagtcccctgtctgaaaaataaataataataataataattgcttcacttacacttcatgtgatcatgttcccaacacttagtttgtcttacaggaaagcttgacagagacttgtgggagcttgatcaagctccttgcttttagataagcaaggattttgatttgattttaaaatgttgtgttgttttgttttgttttttgaggcagggtctcactcctgtcacccaggctggagtgcagtggcatgatcatggttcactgcagcctcaacttcctgagctcaggtgatcctcgtgcctcagcctcccgagtagctggaactacaagtgcatgccaccatgcacttgtaacaataatgttacgt

SEQ ID NO: 13 - DNA, 1080 bp, Homo sapiens
accttgtgctgttaggaatttggtgggtagcttccccatctattttatacttttacatatcacatacacacttacctatatcatatctcaaaaccagataatattgatttctctgtgtttaagttacaaaatgatcactgtaggtattgttctgcagcttactttacataatattatgattttgagctctcttgatatgtgcggatgtaatttattatacttcattgctgtattttgatttataaatatgccacttctttctaatctgtttcctactgatgacagtttggttatttcctgattttttttaactgtaattatttactttcactagtctcctagtgccaatagtatttaaaactaaaattagtctggtttttatgaaccttggcagtgtagtttgagtcttttttcccctacttctgtggactgtctgctcagtgttgtcatgtttcggggttgtagaacatcacacagcgtgttgcttttcgtcctggcaggcagtccttgatggagatgcagagtcgtcatggggcgagaacaagtgactctgaccagcaggcttaccttgttcaaagaggtgagtcccgtgtgatcctggattttcaggaaatagctatcctatgaaaaagatgcttgaagaaaaattccacttcattctctacaatggattccaaatcaaggcaccaaaaatatagcacccgtcagtctcattaccacagcactcccatctccatccattacccaccgaatccagaccagacccttcaccctgccagaaggtgcctggcacggccacactttttcttttttttctttttttttgagacagaatttcgctgtgtcgtccaggctggagtgcagtggcgagatctcggctcactgcaacctccacttcctgtgttcaaacggttctccttccacagcctcccgagtggctggaattacaggcgtgcaccgccacacccagctaatttttgtatttttaatagagatggggtttcaccgtgttggccaggctggtctcgaactcctgacctcaactaacctgcctgtctcggtctcccaaagtaccgggat

SEQ ID NO: 14 - DNA, 1122 bp, Homo sapiens
catgccagtaatcctagcactttgggaggccaaggtgggcagatcatgaggtcaggagttcgagaccagtctggccaacatggcaaaaccacatctctactaaaaatacaaaaattagctgggcgtggtggcgcgcacctgtgatcccagctactcaggaggccaaagcaggaggatcacttgaacctgggaggcggaggttgcagtgagccaagatcgtgccactgccctccagcctgggtgacagcgagactccgtctcaaaaaaaaaaaaaaaaaaaaaaaatcctaaaataatagggaagcaggtatcacttggagagatttttctctatgtgcatcgtgatgacttcagttaaagaccaaacacctgtgctcatgtcccactacgtgttgaatacgaagttgaactgatgttaaaactcgccatctgttcttcaagtgaaacaaacacaactgcctgcaaaatggaactaatggaattatcatacttattcccaggagctgaggacagggactggcggcaacagcggaatattccgattcattcctgccccaagtgtggagaggttctgcctgacatagacacgttacagattcacgtgatggattgcatcatttaagtgttgatgtatcacctccccaaaactgttggtaaatgtcagattttttcctccaagagttgtgcttttgtgttatttgttttcactcaaatattttgcctcattattcttgttttaaaagaaagaaaacaggccgggcacagtggctcatgcctgtaatcccagcactttgggaggtcgaggtgggtggatcacttggggtcagggtttgagaccagcctggccaacatggcggaaccctgtctctaccaaaattacaaaaattagccgagcatggtggcgcatgcctgtagtcgcagctactcgcgaggttgaggcaggagaattgcttgaacccaggaagtggcagttgcagtgagccgagacgacaccactgcactccagcctgggtgacagagggagactctgtctcgaaagaaagaaagaaaaaaaggaaggaaggagaaggaaggaaggagaagaaaaggtacctgttctacgtagaacacctttggtggagt

SEQ ID NO: 15 - DNA, 23 bp, Artificial
  Designed DNA based on OPTN gene
aaggaagaatcaaaaatgtccaa

SEQ ID NO: 16 - DNA, 25 bp, Artificial
  Designed DNA based on OPTN gene
acctgtttccaaaagtaaaaatcag

SEQ ID NO: 17 - DNA, 20 bp, Artificial
  Designed DNA based on OPTN gene
gaaatgatgttcatcccgct

SEQ ID NO: 18 - DNA, 21 bp, Artificial
  Designed DNA based on OPTN gene
cccttaatagggcaaccaatc

SEQ ID NO: 19 - DNA, 20 bp, Artificial
  Designed DNA based on OPTN gene
tgtgccactacacctggcta

SEQ ID NO: 20 - DNA, 26 bp, Artificial
  Designed DNA based on OPTN gene
tttttccattttagctatgataaccc

SEQ ID NO: 21 - DNA, 20 bp, Artificial
  Designed DNA based on OPTN gene
gaaactgaccttcacgcctt

SEQ ID NO: 22 - DNA, 20 bp, Artificial
  Designed DNA based on OPTN gene
gagccaaacaggaaccaaac

SEQ ID NO: 23 - DNA, 19 bp, Artificial
  Designed DNA based on OPTN gene
aggaccagctgtgcttgtt

SEQ ID NO: 24 - DNA, 22 bp, Artificial
  Designed DNA based on OPTN gene
gctacgttgtagatgaacctca

SEQ ID NO: 25 - DNA, 20 bp, Artificial
  Designed DNA based on OPTN gene
tgcatggaaacaaatggtgt

SEQ ID NO: 26 - DNA, 20 bp, Artificial
  Designed DNA based on OPTN gene
cacagagcaggacaaggaca

SEQ ID NO: 27 - DNA, 19 bp, Artificial
  Designed DNA based on OPTN gene
cccagtgcatccaaattga

SEQ ID NO: 28 - DNA, 22 bp, Artificial
  Designed DNA based on OPTN gene
tcatgctcacacattaactgga

SEQ ID NO: 29 - DNA, 20 bp, Artificial
  Designed DNA based on OPTN gene
ttcagaaatatggccaggtc

SEQ ID NO: 30 - DNA, 20 bp, Artificial
  Designed DNA based on OPTN gene
cagagtgcaaacaatcagca

SEQ ID NO: 31 - DNA, 27 bp, Artificial
  Designed DNA based on OPTN gene
gagagtaagaaatgctagtaggtcgtg

SEQ ID NO: 32 - DNA, 21 bp, Artificial
  Designed DNA based on OPTN gene
tccctgaccataggacattca

SEQ ID NO: 33 - DNA, 18 bp, Artificial
  Designed DNA based on OPTN gene
ccactcctgggctcaaac

SEQ ID NO: 34 - DNA, 21 bp, Artificial
  Designed DNA based on OPTN gene
tgccacaaactggaaagaatc

SEQ ID NO: 35 - DNA, 20 bp, Artificial
  Designed DNA based on OPTN gene
cagtgcagttgcaggatttg

SEQ ID NO: 36 - DNA, 21 bp, Artificial
  Designed DNA based on OPTN gene
tgatcaagctcccacaagtct

SEQ ID NO: 37 - DNA, 24 bp, Artificial
  Designed DNA based on OPTN gene
tttcactagtctcctagtgccaat

SEQ ID NO: 38 - DNA, 20 bp, Artificial
  Designed DNA  based on OPTN gene
gattcggtgggtaatggatg

SEQ ID NO: 39 - DNA, 20 bp, Artificial
  Designed DNA based on OPTN gene
gggaagcaggtatcacttgg

SEQ ID NO: 40 - DNA, 18 bp, Artificial
  Designed DNA based on OPTN gene
atccacccacctcgacct